(19) United States
(12) Patent Application Publication
(10) Pub. No.: US 2023/0129345 A1
(43) Pub. Date: Apr. 27, 2023

(54) SYSTEM, METHOD, AND COMPUTER PROGRAM FOR RECOMMENDED MEDICAL TREATMENTS BASED ON DATA MINING

(71) Applicant: Legrande Corporation, Rexburg, ID (US)

(72) Inventors: Anthony Sine, Laguna Beach, CA (US); Malaquis Alex Lopez, Fullerton, CA (US)

(21) Appl. No.: 17/510,166

(22) Filed: Oct. 25, 2021

(51) Int. Cl. G16H 50/20; G16H 50/70

(52) U.S. Cl. CPC G16H 50/20; G16H 50/70

(57) ABSTRACT

A system, method, and non-transitory computer readable storage medium are provided for recommended medical treatments based on data mining. In use, retrieve at least one of historical interaction data of a first patient, or real-time interaction data of the first patient is retrieved. Additionally, at least one potential diagnosis is presented. A selection is received of a determined diagnosis from the at least one potential diagnosis by a medical provider. A selection of additional patient attributes by the medical provider is received. Further, a recommended treatment is presented to the medical provider based, at least in part on data mining: at least one of the historical interaction data of the first patient, or the real-time interaction data of the first patient; the selection of the determined diagnosis; and the selection of additional patient attributes.

### RELATED APPLICATIONS

The present application incorporates by reference the following application by reference in their entirety for all purposes: U.S. application Ser. No. 7/510,192 entitled “SYSTEM, METHOD, AND COMPUTER PROGRAM FOR ASYNCHRONOUS AND SYNCHRONOUS MEDICAL INTERACTIONS BASED ON DATA MINING” filed Oct. 25, 2021 (docket number LEG1P002).

### FIELD OF THE INVENTION

The present invention relates to recommended medial treatments, and more particularly to instructions by a medical provider for medicine or treatment based on data mining.

### BACKGROUND

Currently, medical providers generally follow a consistent treatment based on the diagnosis determined. For example, a diagnosis of bacterial conjunctivitis (i.e. pink eye) may yield a treatment of Tobramycin drops. The recommended treatment may often be based on the medical provider's preference, a historical pattern, a database of recommendations, and/or a recommended course of action based on a recommendation from another provider. Such course of recommendations, however, do not take into account patient specific attributes, which may directly affect the recommended treatment selected.

As such, there is thus a need for addressing these and/or other issues associated with the prior art.

### SUMMARY

A system, method, and non-transitory computer readable storage medium are provided for recommended medical treatments based on data mining. In use, retrieve at least one of historical interaction data of a first patient, or real-time interaction data of the first patient is retrieved. Additionally, at least one potential diagnosis is presented. A selection is received of a determined diagnosis from the at least one potential diagnosis by a medical provider. A selection of additional patient attributes by the medical provider is received. Further, a recommended treatment is presented to the medical provider based, at least in part on data mining: at least one of the historical interaction data of the first patient, or the real-time interaction data of the first patient; the selection of the determined diagnosis; and the selection of additional patient attributes.

### BRIEF DESCRIPTION OF THE DRAWINGS

FIG. 1 illustrates a method for presenting a recommended treatment based on data mining, in accordance with one embodiment.

FIG. 2 illustrates an online network architecture for asynchronous and synchronous medical interactions, in accordance with one embodiment.

FIG. 3 illustrates a system for patient interactions, in accordance with one embodiment.

FIG. 4 illustrates a ranking of medical data, in accordance with one embodiment.

FIG. 5 illustrates user interfaces for interacting with a patient, in accordance with one embodiment.

FIG. 6 illustrates a method for presenting a recommended treatment based on data mining, in accordance with one embodiment.

FIG. 7 illustrates a system for doctor-led interaction, in accordance with one embodiment.

FIG. 8 illustrates system for patient-led communication, in accordance with one embodiment.

FIG. 9 illustrates a network architecture, in accordance with one possible embodiment.

FIG. 10 illustrates an exemplary system, in accordance with one embodiment.

FIG. 11 illustrates an exemplary therapeutic interchange, in accordance with one embodiment.

### DETAILED DESCRIPTION

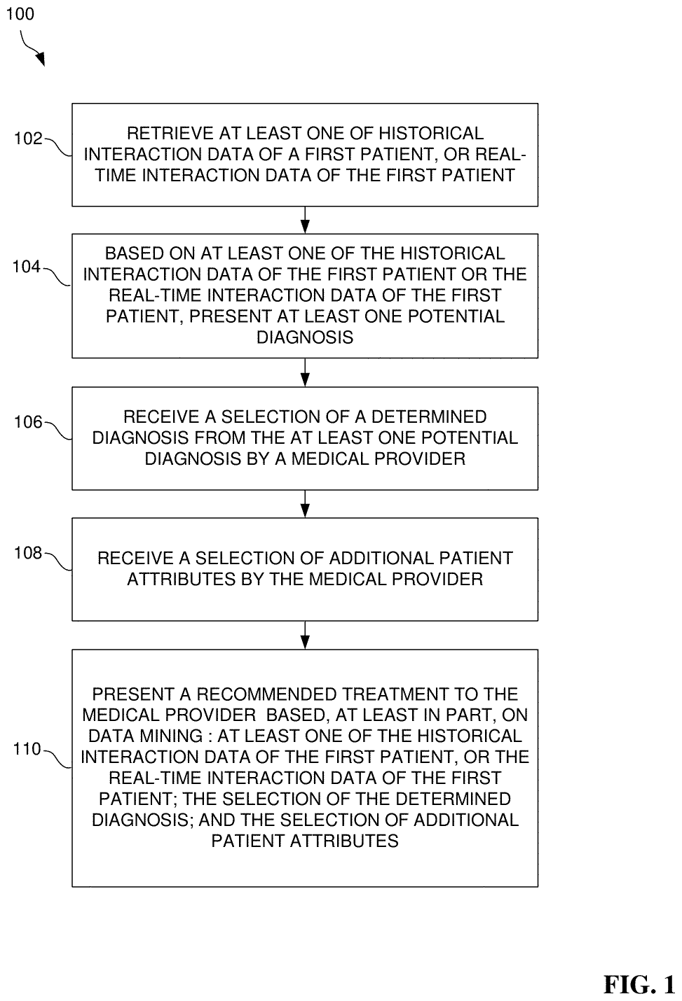

FIG. 1 illustrates a method 100 for presenting a recommended treatment based on data mining, in accordance with one embodiment.

As shown, at least one of historical interaction data of a first patient, or real-time interaction data of the first patient is retrieved. See operation 102. In the context of the present description the historical interaction data may include any asynchronous interaction (i.e. non real-time). For example, historical interaction data may include patient medical history, transmission of content (including but not limited to text, email, voice message) retrieved on demand, lifestyle habits (including but not limited to diet, exercise, tobacco use, alcohol use, etc.), personal data (including but not limited to sex, date of birth, ethnicity, age of when condition was diagnosed if applicable, etc.), remote patient monitoring (based on collected and/or transmitted data), at least one patient action, and/or any other medically related data or medically related action.

In one embodiment, patient medical history may include one or more of: patient and medical provider interaction, patient medical procedure(s), patient medical condition(s), patient medical allergy(ies), family medical procedure(s), family medical condition(s), or family medical allergy(ies), prior medical interaction, diagnosis, prescription(s), illness(es), surgery(ies), immunization(s), results of physical exams and tests, family health history, frequency of past screening, genetic test(s), substance abuse, pregnancy complications, past hospitalizations, etc.

Additionally, the patient action may include one or more of: receipt of medical treatment, refill of a prescription, decision to not take medical action, refusal of medical treatment, election of an alternative treatment, selection of a lower-cost option, response to or sending a communication from/to a medical representative, a change a health insurance plan, fulfillment of medical prescription, completion of medical prescription, tracking of dosage, a request for refill, indication of drug side effect, completion of daily reminder relating to medical prescription, completion of interaction with caregiver or medical provider, tracking of exercise, tracking of diet, tracking of food intake, body vital measurements, body vital statistics, body vital historical data, and/or fulfillment of medical prescription coupon.

In one embodiment, the historical interaction data may be stored on a server (e.g. cloud or server-based electronic medical record system, dedicated medical server, HIPAA-compliant data storage, etc.). In another embodiment, the historical interaction data may be stored locally (e.g. on a patient's device, etc.) and then uploaded to a server at a later time. Additionally, data may be synced between a patient's device and a server (including but not limited to two-way sync, one-way sync, etc.). Such syncing of data may be based on privacy limitations, release of medical records, age of patient (or age of minor patient, etc.), healthcare release of information, etc.

Additionally, within the context of the present description, real-time interaction data may include any synchronous (i.e. real-time) interaction. For example, real-time interaction data may include real-time behavior observations, real-time communication, digital communication, video patient evaluations, health triage, in-person visit/communication (face-to-face communication), live examination, telehealth communication, in-house provider visit, in-office provider visit, current patient and/or vital information (height, weight, symptom(s), temperature, pain, blood pressure, heart rate, etc.), etc. In one embodiment, synchronous medical data may be obtained in-person. In other embodiments, synchronous medical data may be obtained remotely (e.g. via telehealth solutions, video chat, phone call, etc.). The synchronous medical data may be based also on one or more sensing devices (e.g. smart watch, remote vitals monitoring (e.g. body temperature, heart rate, RR interval, respiration rate, oxygen level, etc.), wearables, ambient sensors (e.g. movement, fall detection, sleep cycles, etc.), etc.).

As used herein, the term historical interaction data may be synonymous with asynchronous data within the context of the present description, and the term real-time interaction data may be synonymous with synchronous data within the context of the present description.

Additionally, based on at least one of the historical interaction data of the first patient or the real-time interaction data of the first patient, present at least one potential diagnosis. See operation 104. Additionally, a selection is received of a determined diagnosis from the at least one potential diagnosis by a medical provider. See operation 106. In one embodiment, the doctor (alone) may determine the diagnosis based on the symptoms provided via one or both of the historical interaction date of the first patient or the real-time interaction data of the first patient. In another embodiment, a doctor may input the symptoms (based on one or both of the historical interaction date of the first patient or the real-time interaction data of the first patient) into a medical system which may, in turn, provide possible diagnoses, from which the doctor can then review and select the appropriate diagnosis. As such, in some embodiments, the doctor may be able to data mine one or both of the historical interaction date of the first patient or the real-time interaction data of the first patient in determining a diagnosis. Additionally, in one embodiment, the at least one potential diagnosis may be doctor-led, doctor-directed, and doctor-controlled.

In one embodiment the appropriate diagnosis may be based, at least in part, on clinical data. For example, the doctor may have his/her own patients and may have internal data relating to the best results for patients with similar symptoms (based on a recommendations given to recover). This clinical data may be obtained relating to an internal clinical study (i.e. private, not-public, etc.). Such internal clinic study may include real world evidence of actual anonymized patients, and may be include metadata tags (e.g. date data collected, etc.). Further, the individual data and the collective data of the internal clinic study may be used for purposes of determining the appropriate diagnosis.

Additionally, the clinical data may be obtained relating to an external clinical study (i.e. public, etc.). Such external data may include real world evidence of actual anonymized patients. The sample size of data (for both the internal clinical study and the external clinic study) may surpass any statistically significant threshold. Further, the individual data and the collective data of the external clinic study may be used for purposes of determining the appropriate diagnosis.

Moreover, in determining the appropriate diagnosis, the doctor may additionally receive input from medical data from clinical databases. U.S. application Ser. No. 17/510,192 entitled “SYSTEM, METHOD, AND COMPUTER PROGRAM FOR ASYNCHRONOUS AND SYNCHRONOUS MEDICAL INTERACTIONS BASED ON DATA MINING” filed Oct. 25, 2021 (docket number LEG1P002) discloses in further detail the use of medical data from clinical databases which can be the basis for a doctor recommendation, the content of such application of which is incorporated herein by reference for all purposes.

In various embodiments, the clinical data may include one or more of patient history, medical treatments, effectiveness of a prior course of treatment, a prescription allergy, a length of a recovery, etc.

Further, a selection is received of additional patient attributes by the medical provider. See operation 108. Additional patient attributes may include at least one of pricing considerations, lifestyle considerations, or availability considerations. In one embodiment, the pricing considerations, lifestyle considerations, and/or availability considerations may be inputted in advance by the patient, and/or by a medical provider (e.g. nurse practitioner, doctor, etc.). In another embodiment, the pricing considerations, lifestyle considerations, and/or availability considerations may be inputted and/or selected in real-time with the patient present as part of the real-time interaction with the first patient. If data was previously entered in relation to any of the pricing considerations, lifestyle considerations, and/or availability considerations, the doctor may review such selections with the patient to determine if they are still applicable and/or need to be modified in any manner.

For example, pricing considerations may include the ability for a doctor to know how much a prescription will cost the patient at a patient-selected pharmacy based on the patient's insurance plan. In conventional scenarios, a doctor will often provide a prescription without having any information as to the pricing of such prescription for that particular patient. Thus, the present method provides input to the doctor so that a prescription recommended is given that aligns with the budget and cost constraints of the patient. Additionally, one or more alternatives to the desired prescription (such as generic brands, etc.) may be presented (which again include price determinations that is personalized based on the insurance plan of the patient, the preferred pharmacy of the patient, etc.).

In another embodiment, pricing considerations may include available prescription coupons (or provide the doctor with recommendations to give to the patient for available reward programs to enable greater savings). Additionally, pricing considerations may include patient willingness to pay, manufacturer's rebate(s), pharmacy charges, etc. Further, comparable options and/or alternative options (to a desired course of action and/or remedy) may be provided with pricing details for each option. In this manner, a complete view of the total pricing to a patient may be taken into consideration.

Lifestyle considerations may include any lifestyle restriction of a patient. For example, a patient may have religious constraints, sleep constraints, eating constraints (e.g. vegetarian, vegan, etc.), physical activity (e.g. no knee related activity, etc.), time constraints (e.g. need to work a 6 hour work day, etc.), inability to perform (e.g. need to lay down when receiving a blood test, can't do eye drops, etc.), patient bias, family bias, community constraint, etc. Each of these lifestyle considerations may be selected and/or modified by the doctor as needed.

Availability considerations may include any restriction on availability of the patient, the recommended course of action, the prescription, etc. For example, a patient may need to receive a course of treatment that is available within the next hour versus within the next few days. Timing therefore can be considered. Additionally, in one embodiment, a patient may have a preference to begin treatment as soon as possible (even if it is elective). Further, pharmacy, wholesalers, and manufacturers may each have different levels of availability. Knowing the availability (on timing) of when a recommended course of action is needed may dictate the source from which the prescription is obtained, as well when such can be expected to be received from such source. Additionally, in one embodiment, availability considerations may include one or more of: location of the first patient with respect to a location of a pharmacy, stocking of prescriptions at a preferred patient location.

Furthermore, a recommended treatment is presented to the medical provider based, at least in part, on data mining: at least one of the historical interaction data of the first patient, or the real-time interaction data of the first patient; the selection of the determined diagnosis; and the selection of additional patient attributes. See operation 110. For example, the recommended treatment may include a clinical practice guideline, one or more medical actions (e.g. to receive treatment, cancer screening, follow up care, etc.), lifestyle change (e.g. get more sleep, eat less sugar, smoke less, get more exercise, etc.), a prescription (e.g. drug, medicine, etc.), etc.

In one embodiment, data of the patient attributes, data of the internal clinic study, data of the external clinic study, the historical interaction data of the first patient, the real-time interaction data of the first patient, and/or any data associated with the patient in any degree, may be ranked. For example, in determining the appropriate recommended treatment, the historical interaction data of the first patient and/or the real-time interaction data of the first patient may be ranked higher than, e.g., the data of the external clinic study, the data of the internal clinic study, and/or the patient attributes.

In one embodiment, the doctor may view the recommended treatment as a compilation display. For example, a legend with possible available products, and all of the criteria for the patient attributes may be displayed such that the doctor could modify any of the foregoing in real time to modify the display of recommended treatments. Further, the doctor may modify and/or refine any of the historical interaction data, the real-time interaction data, the data of the external clinic study, and/or the data of the internal clinic study, which in turn may continually refine the display of recommendation treatments.

In this manner, the recommended treatment may be a fluid display of results based on any of the inputs (patient attributes, available products, the historical interaction data, the real-time interaction data, the data of the external clinic study, and/or the data of the internal clinic study) retrieved for or inputted in response to the current real-time interaction session.

In various embodiments, the method 100 may be implemented in a variety of scenarios. For example, doctors are often blind as to whether the prescribed course of action is even applicable to the present patient. For example, the patient may restrict their consumption of animal-derived products, and the doctor may unknowingly prescribe a medicine that includes active or inactive ingredients derived from a prohibited source. As such, the method 100 may be used to resolve such discrepancy. The doctor can input (consistent with operation 105) a patient attribute (e.g. the patient has a vegan diet and constraint, etc.) which may remove those prescriptions and/or medical options available as a recommended course of treatment. In some instances, if the patient attributes are overly restrictive (such that no recommended treatment can be provided), the doctor may rank which of the patient attributes is most important to the patient so that a recommended treatment can be provided in a manner least impactful to the patient's preferred attributes.

Additionally, the recommended treatment (consistent with the operation 110) may be presented as a result of data mining available data groups (the historical interaction data, the real-time interaction data, the data of the external clinic study, and/or the data of the internal clinic study), as well as additional filters (e.g. patient attributes, availability of products, etc.).

As an example, an initial diagnosis may be provided by a doctor. Such diagnosis may be based on the patient indicated past symptoms and/or medical conditions, and current symptoms. Additionally, the doctor may conduct an evaluation to verify the current symptoms. The doctor, in one embodiment, may determine that the patient has a sinus infection and indicates the diagnosis into the system. In another embodiment, the doctor may input the past symptoms/medical conditions and current symptoms into the system and retrieve a list of possible diagnosis. Once the diagnosis has been selected, the doctor may then tailor the recommended course of treatment based on the products available, and preferences of the patient (criteria for filters). In this manner, the recommended course of treatment is personalized according to the specific needs of the patient, rather than a one size fits all treatment approach based on similar symptoms.

In one embodiment, after deciding upon a recommended treatment, the doctor may then submit the prescription based on the input of the patient. In one embodiment, the submission may be to a local preferred pharmacy. In another embodiment, the submission may be to an online prescription service, which in turn, may send out the prescription to the patient. In one embodiment, the prescription originating from the doctor may include the ability to e-sign the prescription, and/or submit the prescription to the recipient (e.g. pharmacy, online prescription service, etc.) for fulfillment via a digital prescription.

Of course, it is to be appreciated that the method 100 may be implemented as appropriate in each state and jurisdiction based on the local legal requirements as it relates to pharmacy, prescriptions, and/or medical interactions. Further, the method 100 may be implemented in a manner to be compliant with laws of each state and jurisdiction.

As an example, a patient may be suffering from farsightedness. The patient may have an exam with an eye doctor to diagnose the issue. Such an analysis may include receiving historical interaction data (including any family eye issues, prior eye surgeries, prior eye tests, genetic disposition, etc.) and real-time interaction data (based on discussion, based on live inspection, based on sight test, etc.). The doctor may receive clinical data results indicating that others with similar historical and real-time interaction data are often suffering from presbyopia. The doctor may determine that the patient is indeed suffering from presbyopia and indicate that a recommended treatment would include eye drops. The patient may provide input that their prescription insurance recently changed, and the location of their preferred pharmacy changed. The doctor may input such information as patient attributes to then determine which pharmacy near the patient would allow the patient to obtain the prescription at the lowest cost. The doctor may also determine whether the patient wishes to receive the eye drops by mail, and may set the patient up on a refill service, such that when the patient begins to run out (based on rate of consumption of the eye drops based on the prescribed course of treatment), the prescription may be automatically fulfilled and sent to the patient. After completing the recommended treatment, the patient may receive a notification of what was discussed with the doctor, as well as an option to pay for the eye drops, whereupon, the patient may then be sent shipping information that can be tracked by the patient (as well as the doctor). In this manner, the doctor may be able to diagnosis the issue based on data mining the historical interaction data, the real-time interaction data, and the clinical data. Additionally, the doctor may further present a preferred treatment based on additional attributes as dictated by the patient. As such, the medical experience by the patient with the doctor may be personalized such that the patient receives a personalized tailoring of both the diagnosis and the recommended treatment.

In one embodiment, the recommended treatment to the medical provider may be further presented based on input from an artificial intelligence system that analyzes at least two of the historical interaction data of the first patient, the real-time interaction data of the first patient, the selection of the determined diagnosis, or the selection of additional patient attributes.

More illustrative information will now be set forth regarding various optional architectures and uses in which the foregoing method may or may not be implemented, per the desires of the user. It should be strongly noted that the following information is set forth for illustrative purposes and should not be construed as limiting in any manner. Any of the following features may be optionally incorporated with or without the exclusion of other features described.

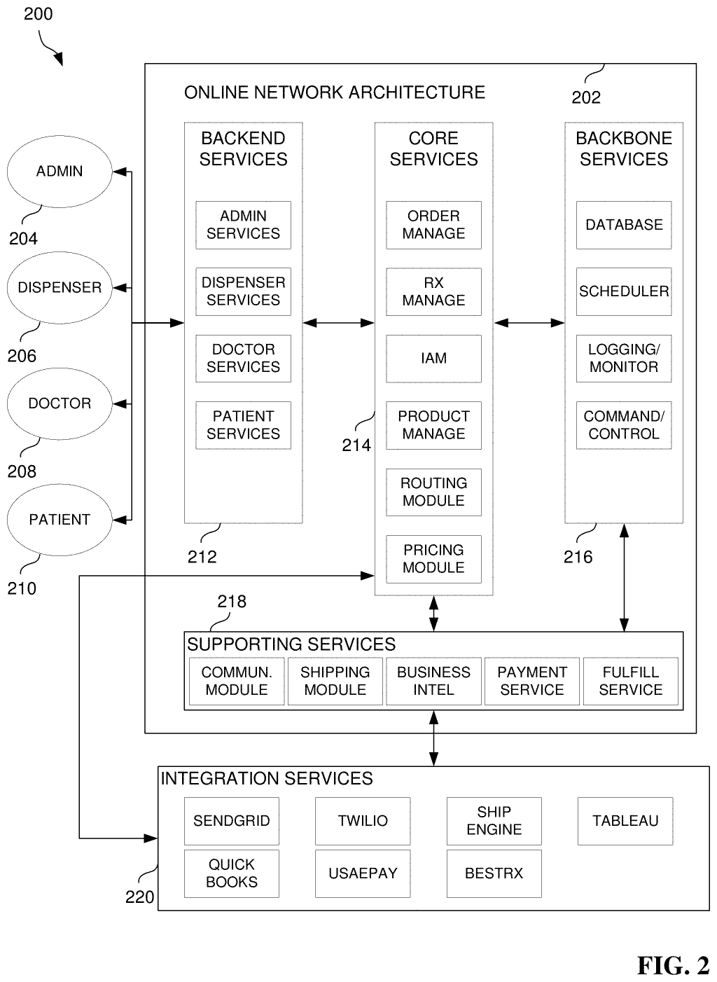

FIG. 2 illustrates an online network architecture 200 for asynchronous and synchronous medical interactions, in accordance with one embodiment. As an option, the online network architecture 200 may be implemented in the context of any one or more of the embodiments set forth in any previous and/or subsequent figure(s) and/or description thereof. Of course, however, the online network architecture 200 may be implemented in the context of any desired environment. Further, the aforementioned definitions may equally apply to the description below.

As shown, the online network architecture 200 is connected (individually and/or collectively, as needed) to an admin 204, a dispenser 206, a doctor 208, and/or a patient 210. In various embodiments, the admin 204 may assist with managing and/or tracking patient medical interactions, the dispenser 206 may assist with providing resources (including prescriptions) based on the recommendation of the doctor 208, and the doctor 208 may assist with providing the medical interaction with the patient 210. Any or all of the admin 204, the dispenser 206, the doctor 208, and/or the patient 210 may interact with the online network architecture 202.

The online network architecture 202 may include backend services 212, core services 214, backbone services 216, and supporting services 218. The backend services 212 may include admin services, dispenser services, doctor services, patient services, and/or any other service which may facilitate front-end delivery of data and/or services. For example, in one embodiment, a user interface may exist for each of the admin 204, the dispenser 206, the doctor 208, and/or the patient 210 as provided by the backend services 212 for frontend interaction. Additionally, each of the services within the backend services 212 may interact. For example, in response to input being received via the doctor services, the input may be sent (at least in part) to the dispenser services to manage fulfillment of a recommendation provided by the doctor 208.

Additionally, the core services 214 may include order management, prescription management, IAM management (including identity access management), product management, routing module, pricing module, and/or any service that relates to that which is essential for the medical interaction. For example, the IAM management may provide identify services for the patient services of the backend services 212. Additionally, in response to input received by the doctor services of the backend services 212, order management and/or prescription management of the core services 214 may be invoked to assist with managing and tracking a prescription associated with the patient 210. Further, the core services 214 may interact with the supporting services 218 and/or the integration services 220 as needed.

The backbone services 216 may include one or more database(s), a scheduler, logging and monitoring, command and control, as well as any other infrastructure necessary for any of the core services 214, the backend services 212, and/or the supporting services 218. In one embodiment, the backbone services 216 include an interconnect (or a path to connect) between one or more other services. For example, order management of the core services 214 may use the scheduler of the backbone services 216 to schedule delivery of an order. In like manner, the other services of the backbone services 216 may provide functionality to enable completion of one or more other services.

The supporting services 218 may include a communication module, a shipping module, a business intelligence module, a payment service, a fulfillment service, and/or any other service necessary to enable any of the backend services 212, the core services 214, and/or the backbone services 216. For example, in one embodiment, when an order is received via the order management of the core services 214, the scheduler of the backbone services 216 may be invoked (as discussed herein), and the shipping module of the supporting services 218 may be invoked, as well as any further service of the integration services 220 (such as the ShipEngine service to line up shipping of the order). As another example, the business intelligence service of the supporting services 218 may provide enterprise processes to integrate and manage various services (such as, in one embodiment, the scheduler of the backbone services 216, the financial services such as QuickBooks of the integration services 220, data visualization services such as tableau of the integration services 220, and/or the database of the backbone services 216). Of course, any specific service of the supporting services may be connected to various components of any of the backend services 212, the core services 214, the backbone services 216, and/or the integration services 220.

The integration services 220 may include a variety of tasks including financial services, shipping services, revenue services, data management, data extraction, data visualization, etc. In various embodiments, the integration services 220 may include SendGrid, Twilio, ShipEngine, Tableau, QuickBooks, USAEPay, and/or BestRX. Of course, any other integration service may be included within the integration services 220 based on the demands and needs of the online network architecture 202.

In various embodiments, the online network architecture 202 may be flexible such that the platform can be adjusted to meet and evolve with a change of business model or a market need. Additionally, the online network architecture 2020 may provide an intuitive user experience such that use of the system may be simple to learn (from a management point of view of all stakeholders), and patients can engage with the system in a logical manner, which in turn, can increase customer retention.

Further, in one embodiment, the online network architecture 202 may be agile such that each part of the system may be managed and changed independently. This aspect may assist with reducing cost to develop, and with improving a speed to market in providing services. Additionally, given that each service is managed independently, any risk or issue may be isolated to a specific service, thereby increasing run time for all other services.

Still yet, in another embodiment, the online network architecture 202 allows for integration of data such that the system can connect to multiple applications simultaneously (e.g. such as through API links, etc.). Further the data may be pulled at any time for analytics and machine learning.

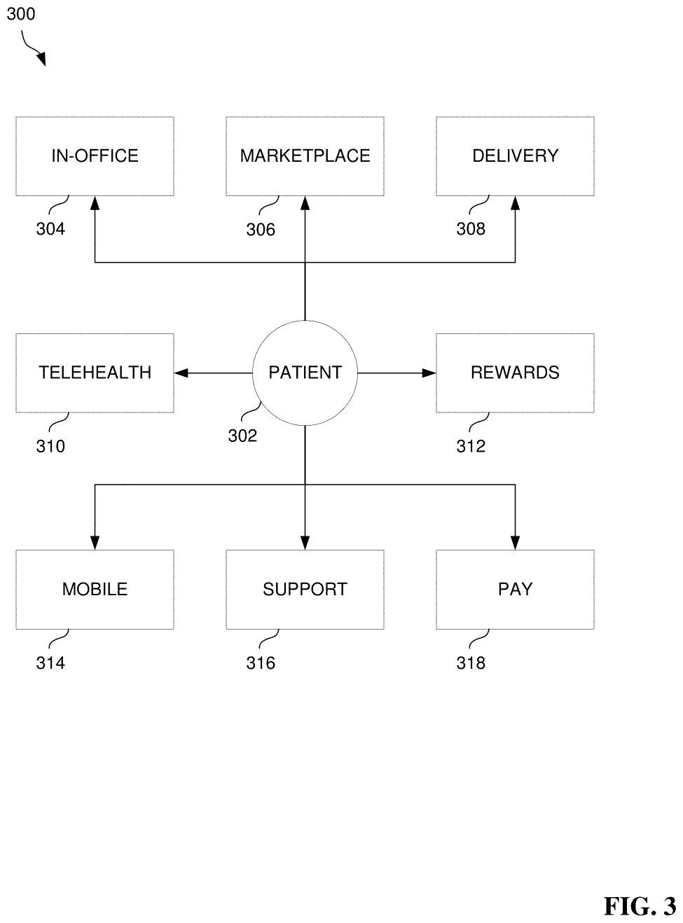

FIG. 3 illustrates a system 300 for patient interactions, in accordance with one embodiment. As an option, the system 300 may be implemented in the context of any one or more of the embodiments set forth in any previous and/or subsequent figure(s) and/or description thereof. Of course, however, the system 300 may be implemented in the context of any desired environment. Further, the aforementioned definitions may equally apply to the description below.

As shown, a patient 302 remains in the center of all interactions. The patient 302 may interact via in-office 304 (e.g. in person medical interaction), medical insurance marketplace 306, delivery 308 (e.g. of prescriptions, of medically-related recommendation, etc.), rewards 312 (e.g. Medicare rewards, completion of healthy activities, completion of financial incentives, etc.), payment 318 (e.g. to pay for medically related bill, to automate medical subscription service, to automate interaction with insurance carrier for processing of bills, etc.), support 316 (e.g. with a medical service provider, etc.), mobile 314 (e.g. mobile delivery service, mobile medical services, etc.), and/or telehealth 310 (e.g. online medical interaction, etc.).

Any or all of the interactions of the system 300 may be tracked as desired and set by the patient 302. For example, the patient 302 may choose to interact with a medical provider via an in-office 304 interaction, but choose not to interact via telehealth 310. Additionally, data associated with a medical interaction may be tracked as desired and set by the patient 302. In this manner, privacy and personal settings may be associated with the interaction and with data resulting from the interaction.

Further, data may exist or be created in relation to any or all of in-office 304, medical insurance marketplace 306, delivery 308, rewards 312, payment 318, support 316, mobile 314, and/or telehealth 310. Such data may be controlled by the patient 302 such that the patient 302 remains at the center of all interactions, all data, and all privacy/securityassociated with the data. In view of such, the system 300 shows a patient-focused, patient-led, and patient-controlled series of interactions that remain under the direction of the patient 302.

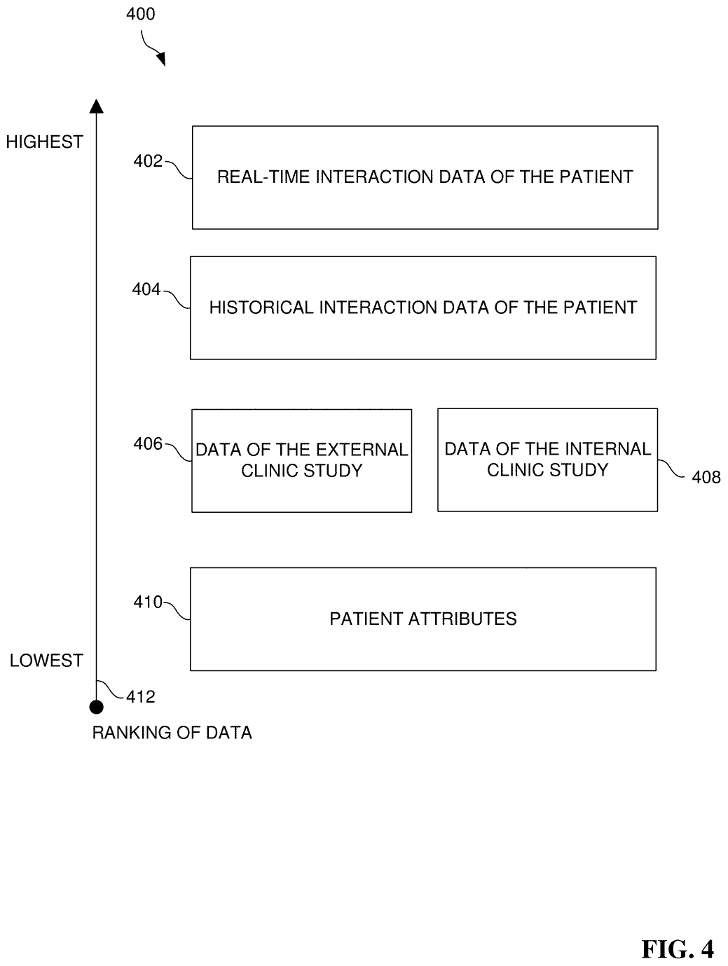

FIG. 4 illustrates a ranking 400 of medical data, in accordance with one embodiment. As an option, the ranking 400 may be implemented in the context of any one or more of the embodiments set forth in any previous and/or subsequent figure(s) and/or description thereof. Of course, however, the ranking 400 may be implemented in the context of any desired environment. Further, the aforementioned definitions may equally apply to the description below.

As shown, the ranking 400 includes real-time interaction data of the patient 402, historical interaction data of the patient 404, data of the external clinic study 406, data of the internal clinic study 408, and patient attributes 410. The ranking is displayed on the scale 412 of ranking data from lowest to highest.

In one embodiment, the real-time interaction data of the patient 402 may be ranked higher than the historical interaction data of the patient 404. In like manner, any of the data 402-410 may be shifted as directed by the doctor, and in view of input provided by the patient.

For example, in one embodiment, the patient may indicate that, due to recent job losses, they would be unable to pay for any medicine more than $50. That particular input (i.e. limitation of $50 for medical treatment) may be inputted and ranked highest so that all resulting recommended treatments are displayed inheriting such condition.

In this manner, the doctor and/or the patient may control the ranking of data 402-410 such that the resulting recommended treatment (in a manner consistent with operation 110) reflects the ranking.

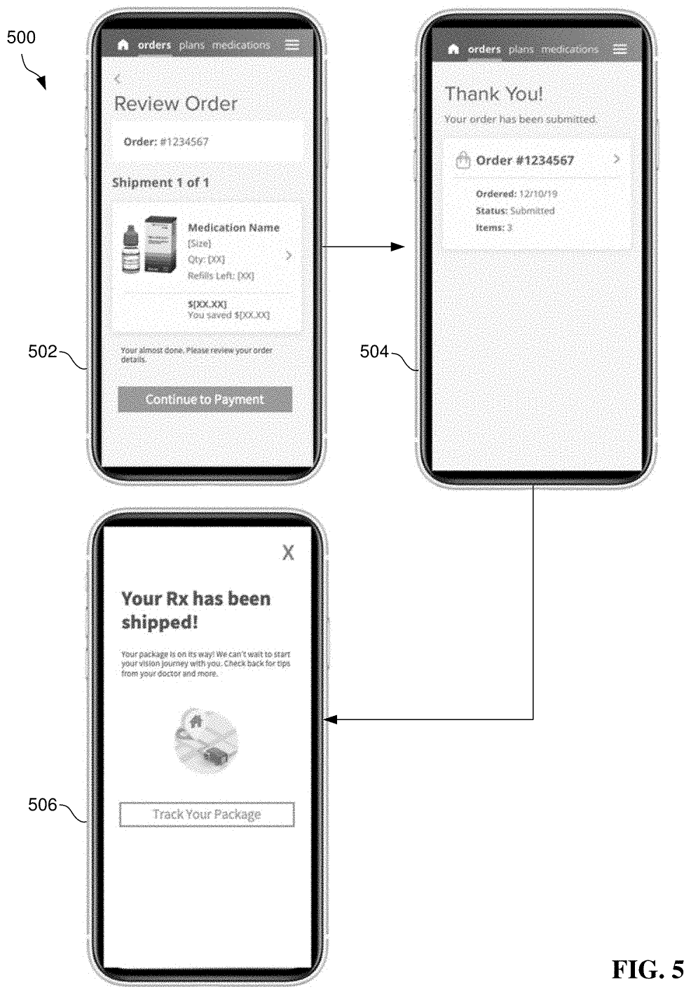

FIG. 5 illustrates user interfaces 500 for interacting with a patient, in accordance with one embodiment. As an option, the user interfaces 500 may be implemented in the context of any one or more of the embodiments set forth in any previous and/or subsequent figure(s) and/or description thereof. Of course, however, the user interfaces 500 may be implemented in the context of any desired environment. Further, the aforementioned definitions may equally apply to the description below.

As shown, user interfaces 500 includes interface 502 which provides the patient with a notification of the order that relates to the agreed upon recommended treatment. Next, in interface 504, a confirmation of the order (after payment) is provided. Next, in interface 506, the ability to track the prescription after it is mailed is provided.

In one embodiment, the user interfaces 500 may be displayed in response to presenting a recommended treatment (consistent with the operation 110). As such, user interfaces 500 display, in one embodiment, a scenario where the patient decided to receive the prescription by mail. As such, the doctor may have submitted the prescription order electronically (digitally) to the prescription provider, and the system, in turn, can manage the tracking of the prescription until it is successfully received by the patient. Once the patient has received such prescription, the system may additionally track further appropriate actions, including refills, as detailed in U.S. application Ser. No. 17/510,192entitled “SYSTEM, METHOD, AND COMPUTER PROGRAM FOR ASYNCHRONOUS AND SYNCHRONOUS MEDICAL INTERACTIONS BASED ON DATA MINING” filed Oct. 25, 2021 (docket number LEG1P002) the content of which is incorporated herein by reference for all purposes.

As such, order management, as prescribed by the doctor, and fulfilled by a pharmacy provider, may be presented (as shown in user interfaces 500) in a transparent manner such that doctor-led recommendations can be tracked by the patient, and the doctor can verify when such prescription was delivered to the patient.

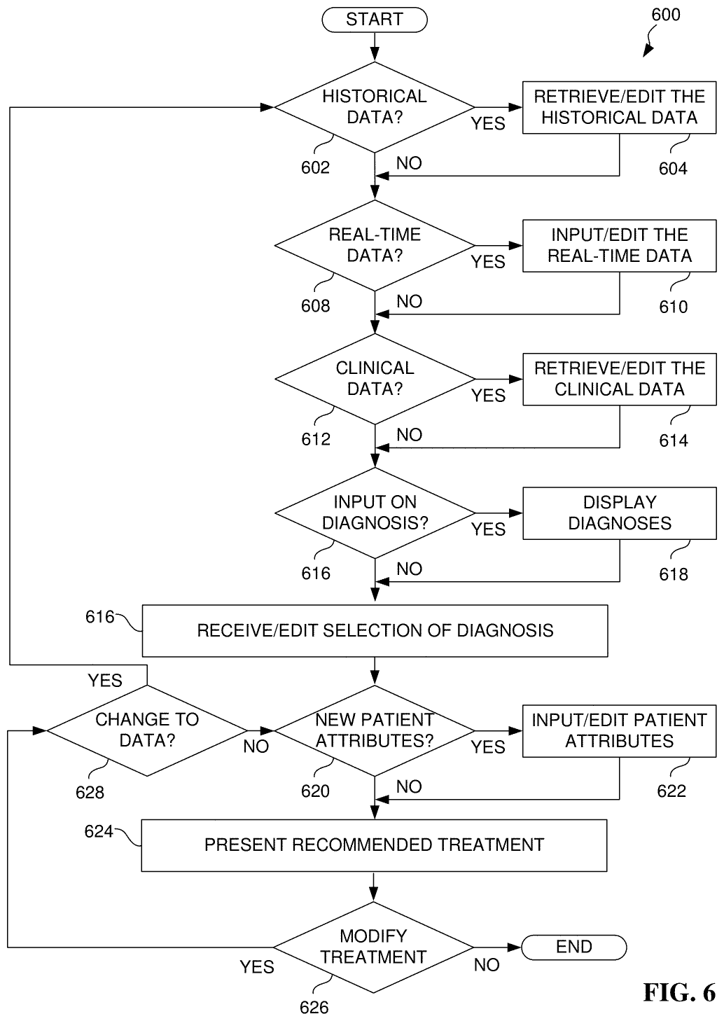

FIG. 6 illustrates a method 600 for presenting a recommended treatment based on data mining, in accordance with one embodiment. As an option, the method 600 may be implemented in the context of any one or more of the embodiments set forth in any previous and/or subsequent figure(s) and/or description thereof. Of course, however, the method 600 may be implemented in the context of any desired environment. Further, the aforementioned definitions may equally apply to the description below.

As shown, the method 600 begins by determining whether historical data for the patient exists. See decision 602. In one embodiment, the historical data may include data as described herein (see, e.g., FIG. 1), and may also include any or all of any of the previously inputted pricing considerations, lifestyle considerations, and/or the availability considerations. Of course, any information relating to the patient may be obtained as part of the historical data. If historical data exists, then it is retrieved and/or edited. See operation 604. For example, if the past historical data was inputted incorrectly, or needs to be modified in any manner, the doctor can make the edits as needed.

Next, it is determined whether real-time data for the patient exists. See decision 606. In one embodiment, the real-time data may include data as described herein (see, e.g., FIG. 1), and may include any real-time information inputted by the doctor. Such real-time information may include current vitals (e.g. of the patient), observed conditions (e.g. a cough, etc.), information from sensors (e.g. oxygen level, etc.), mental state, and/or any other information obtained by the doctor in real-time with the patient. If real-time data exists, then it is inputted and/or edited. See operation 610. For example, if the real-time data was inputted incorrectly, or needs to be modified (or added to) in any manner, the doctor can make the edits as needed.

It is then determined whether clinical data exists. See decision 612. In one embodiment, such clinical data may include data as described herein (see, e.g., FIG. 1). In another embodiment, the doctor may determine that although clinical data may exist, it may not be needed for the present inquiry (and may subsequently bypass this decision). If clinical data exists, then it is retrieved and/or edited. See operation 614. For example, if the clinical data was inputted incorrected, or needs to be modified (or added to) in any manner, the doctor can make the edits as needed.

Further, it is determined whether input is needed to determine the diagnosis. See decision 616. For example, in one embodiment, the doctor may wish for input on what the possible diagnosis is based on the present symptoms. In another embodiment, a clinic requirement may be to submit the gathered symptoms for purposes of receiving back the latest guidance (e.g. from clinical boards) on the recommended course of action. If input is needed in relation to the diagnosis, the diagnosis is displayed. See operation 618. In one embodiment, the display of the diagnosis may include a listing of possible diagnoses (with a confidence score associated with each). A selection of the diagnosis may be received and/or edited. See operation 616. For example, the doctor may select a first diagnosis, and then upon further inquiry and analysis, may determine an alternative (or more precise) diagnosis of the issue.

Additionally, it is determined whether new patient attributes exist. See decision 620. For example, a patient may have had a change of lifestyle, pricing, availability (or never inputted such information before). If new patient attribute data exists, they can be inputted and/or edited. See operation 622. For example, the patient may have the preference of beginning treatment today (indicated as an attribute), but the doctor may update the attribute such that the treatment can be begun within 24 hours (based on input from the patient). In this manner, therefore, the patient attributes can be modified, added to, and/or removed as needed. It is to be appreciated that the historical data (consistent with the decision 602) may include patient attributes previously submitted, and the patient attribute data (per the decision 620) may include input of additional or new information.

A recommended treatment is presented. See operation 624. This recommended treatment may include a preferred treatment, and/or a listing of recommended treatments for review by the doctor.

It is determined whether the treatment needs to be modified. See decision 626. If so, then it is determined whether a change to data (such as the historical data, the real-time data, and/or the clinical data) is needed, or whether the change is limited to just the patient attributes. If the change(s) needed relates to just the patient attributes, then per decision 626, the method returns back to decision 620 (where in operation 622, the patient attributes can be modified). If it is determined that a change is needed to any of the data (such as the historical data, the real-time data, and/or the clinical data), then per decision 628, the method returns back to decision 602.

Once the treatment is decided upon, then the method 600 ends.

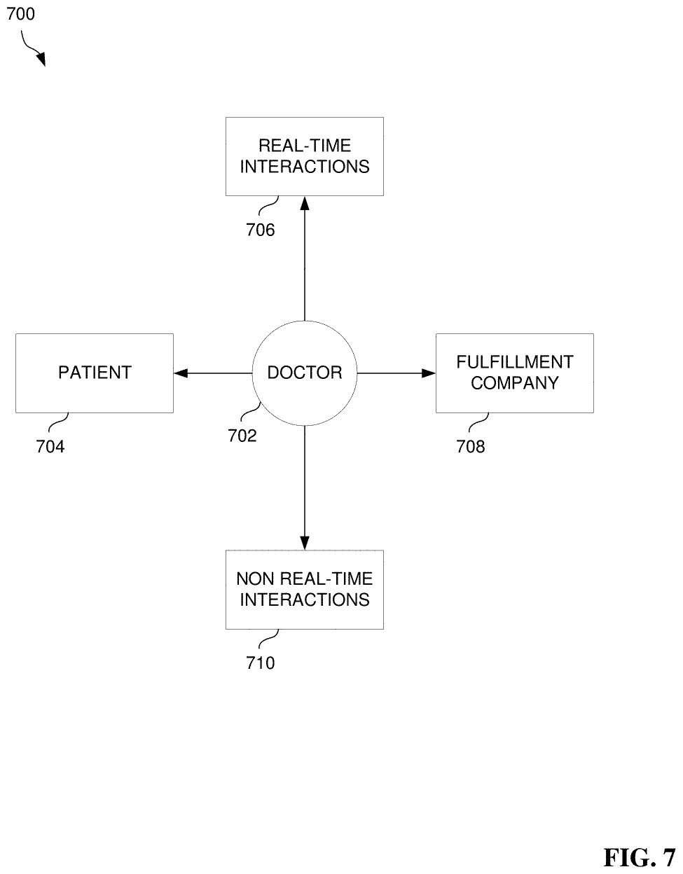

FIG. 7 illustrates a system 700 for doctor-led interaction, in accordance with one embodiment. As an option, the system 700 may be implemented in the context of any one or more of the embodiments set forth in any previous and/or subsequent figure(s) and/or description thereof. Of course, however, the system 700 may be implemented in the context of any desired environment. Further, the aforementioned definitions may equally apply to the description below.

As shown, the system 700 includes a doctor 702 who may interact with a patient 704 and with a fulfillment company 708. The doctor 702 may also engage in real-time interactions 706 (synchronous interactions) and with non real-time interactions 710 (i.e. asynchronous interactions). In one embodiment, the doctor-led interaction may include an omni-channel experience (i.e. more than one channel, etc.) that allows the doctor 702, the patient 704 and the fulfillment company 708 to personalize each experience and compliance. Additionally, the doctor-led interaction may occur in office, in box, and at home with email, text, chat, phone, video, etc. As such, the doctor-led interaction may allow the doctor to more accurately predict the patient need(s) (based on additional data feedback and interaction) and modify a course of action to the patient 704 based on interactions (such as the real-time interactions 706 and/or the non real-time interactions 710). In one embodiment, the fulfillment company 708 may include, but not be limited to, a pharmacy.

Additionally, the fulfillment company 708 may be any resource prescribed to the patient 704 in relation to a recommended course of action. For example, the fulfillment company 708 may include a pharmacy, a physical therapy provider, post-discharge follow-up provider, a hospital, a telehealth provider, and/or any other resource that may enable the patient 704 to fulfill a recommended course of action from the doctor 702.

In one embodiment, the patient 704 may reach out to the fulfillment company 708 (such as a pharmacy) for assistance in diagnosing an issue based on symptoms. This may lead to an asynchronous interaction with the doctor 702 (referred to by the fulfillment company 708), whereupon the doctor 702 can provide recommendations to the patient 704 (e.g. via SMS messaging, via email, etc.). The patient 704 may provide feedback to the doctor 702. Eventually this interaction may lead, if needed, to a synchronous interaction (i.e. live exam) with the doctor 702 to assist the patient 704 in their recovery. The doctor 702 may use the fulfillment company to assist the patient 704 with any resources needed for their recovery.

In another embodiment, interaction between the doctor 702, the fulfillment company 708, and the patient 704 may be based on a subscription or membership arrangement. For example, the patient 704 may pay a monthly amount for medical interactions, which may give the patient 704 access to the doctor 702 and the fulfillment company 708 as needed. Such a membership may include initial diagnosis services, ongoing care services, asynchronous interactions, as well as synchronous interactions.

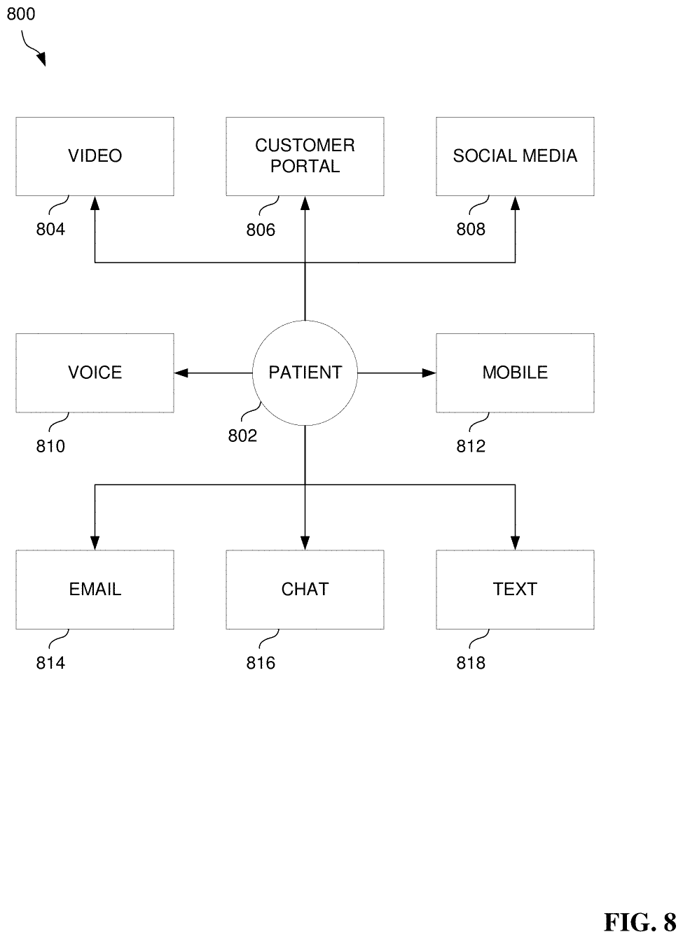

FIG. 8 illustrates a system 800 for patient-led communication, in accordance with one embodiment. As an option, the system 800 may be implemented in the context of any one or more of the embodiments set forth in any previous and/or subsequent figure(s) and/or description thereof. Of course, however, the system 800 may be implemented in the context of any desired environment. Further, the aforementioned definitions may equally apply to the description below.

As shown, the system 800 includes a patient 802 at the center of all types of communication, including video 804, customer portal 806, social media 808, mobile 812, text 818, chat 816, email 814, and/or voice 810. In various embodiments, the customer portal 806 may include a doctor or clinic website or user interface for interacting with a patient.

In one embodiment, the patient 802 remains in control of all forms of communication. Additionally, data may be associated with each of video 804, customer portal 806, social media 808, mobile 812, text 818, chat 816, email 814, and voice 810. Such data may be additionally controlled (including dissemination of the data back to the medical provider) by the patient 802.

Additionally, data from a first source of communication may lead to data obtained from a second source of communication. For example, a telehealth communication (via, e.g., a video 804) may include information on a recommended course of action, which in turn may be followed up with a reminder by email of the recommended course of action, and a subsequent follow up on whether the course of action was completed. Regardless of the form of communication, the patient 802 may remain in control of the data from all communication sources.

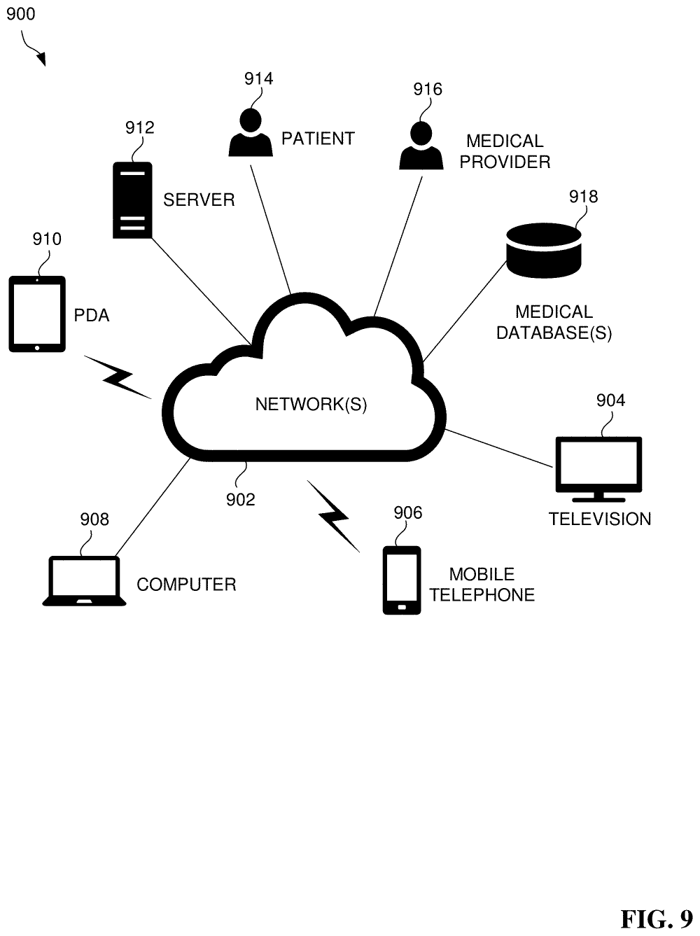

FIG. 9 illustrates a network architecture 900, in accordance with one possible embodiment. As shown, at least one network 902 is provided. In the context of the present network architecture 900, the network 902 may take any form including, but not limited to a telecommunications network, a local area network (LAN), a wireless network, a wide area network (WAN) such as the Internet, peer-to-peer network, cable network, etc. While only one network is shown, it should be understood that two or more similar or different networks 902 may be provided.

Coupled to the network 902 is a plurality of devices. For example, a server computer 912 and an end user computer 908 may be coupled to the network 902 for communication purposes. Such end user computer 908 may include a desktop computer, lap-top computer, and/or any other type of logic. Still yet, various other devices may be coupled to the network 902 including a personal digital assistant (PDA) device 910, a mobile phone device 906, a television 904, etc. Additionally, medical related database(s) 918 may be coupled to the network 902.

Further, coupled to the network 902 may include any number of entities, including but not limited to, patient 914, and medical provider 916. Although not shown, it is to be understood that any entity and number of entities may be connected to the network 902. For example, a pharmacy provider, an in-home provider, a telehealth provider, may in like manner be connected to the network 902.

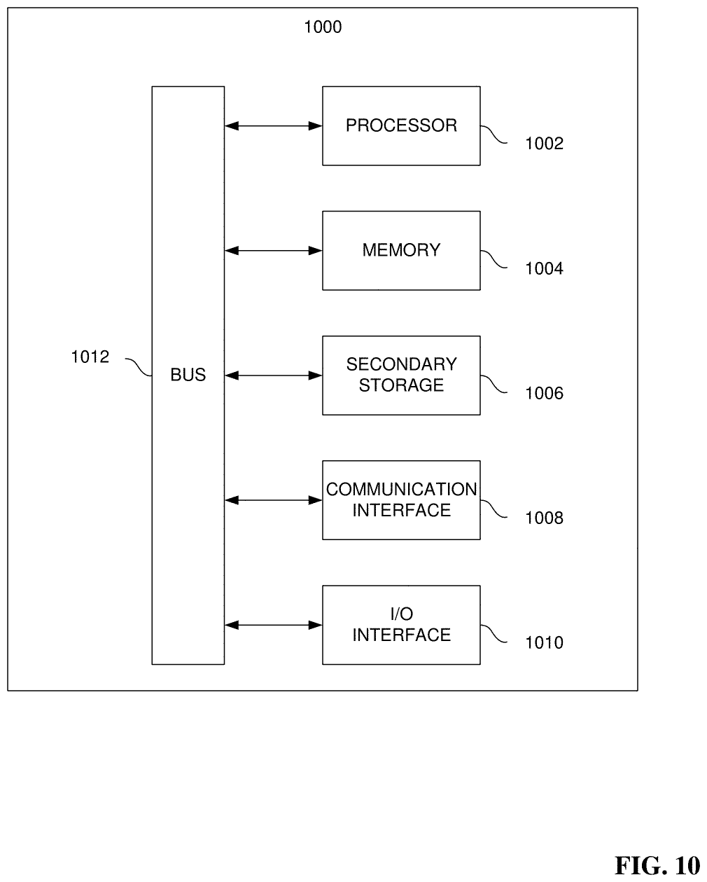

FIG. 10 illustrates an exemplary system 1000, in accordance with one embodiment. As an option, the system 1000 may be implemented in the context of any of the devices of the network architecture 900 of FIG. 9. Of course, the system 1000 may be implemented in any desired environment.

As shown, a system 1000 is provided including at least one central processor 1002 which is connected to a communication bus 1010. The system 1000 also includes main memory 1004 [e.g. random access memory (RAM), etc.]. The system 1000 also includes a graphics processor 1008 and a display 1010.

The system 1000 may also include a secondary storage 1006. The secondary storage 1006 includes, for example, a hard disk drive and/or a removable storage drive, representing a floppy disk drive, a magnetic tape drive, a compact disk drive, etc. The removable storage drive reads from and/or writes to a removable storage unit in a well known manner.

Computer programs, or computer control logic algorithms, may be stored in the main memory 1004, the secondary storage 1006, and/or any other memory, for that matter. Such computer programs, when executed, enable the system 1000 to perform various functions (as set forth above, for example). Memory 1004, storage 1006 and/or any other storage are possible examples of non-transitory computer-readable media. It is noted that the techniques described herein, in an aspect, are embodied in executable instructions stored in a computer readable medium for use by or in connection with an instruction execution machine, apparatus, or device, such as a computer-based or processor-containing machine, apparatus, or device. It will be appreciated by those skilled in the art that for some embodiments, other types of computer readable media are included which may store data that is accessible by a computer, such as magnetic cassettes, flash memory cards, digital video disks, Bernoulli cartridges, random access memory (RAM), read-only memory (ROM), and the like.

As used here, a “computer-readable medium” includes one or more of any suitable media for storing the executable instructions of a computer program such that the instruction execution machine, system, apparatus, or device may read (or fetch) the instructions from the computer readable medium and execute the instructions for carrying out the described methods. Suitable storage formats include one or more of an electronic, magnetic, optical, and electromagnetic format. A non-exhaustive list of conventional exemplary computer readable medium includes: a portable computer diskette; a RAM; a ROM; an erasable programmable read only memory (EPROM or flash memory); optical storage devices, including a portable compact disc (CD), a portable digital video disc (DVD), a high definition DVD (HD-DVD™), a BLU-RAY disc; and the like.

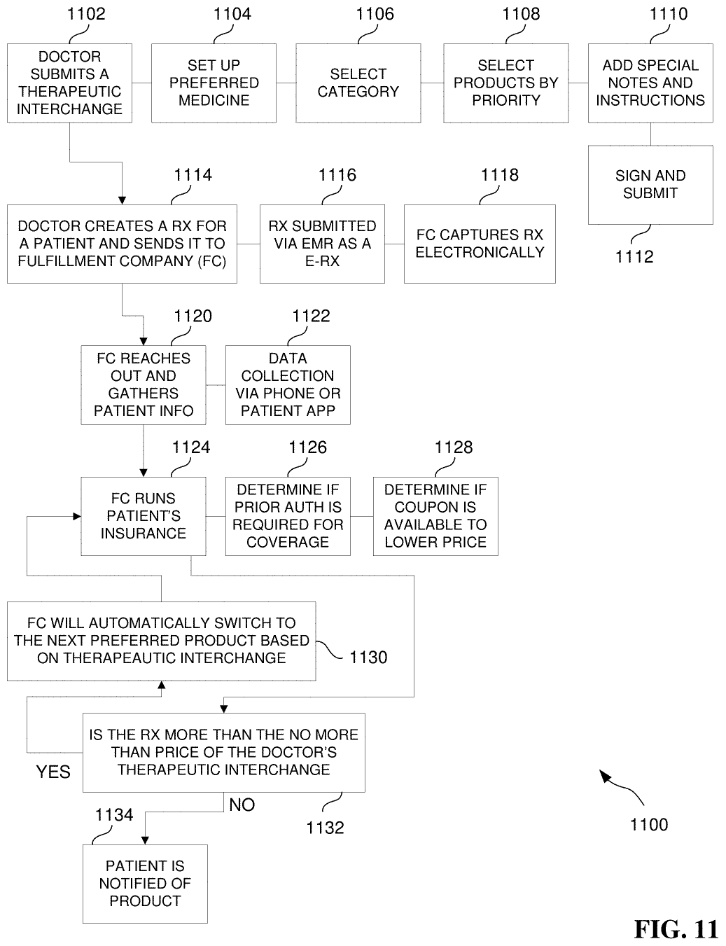

FIG. 11 illustrates an exemplary therapeutic interchange 1100, in accordance with one embodiment. As an option, the exemplary therapeutic interchange 1100 may be implemented in the context of any one or more of the embodiments set forth in any previous and/or subsequent figure(s) and/or description thereof. Of course, however, the exemplary therapeutic interchange 1100 may be implemented in the context of any desired environment. Further, the aforementioned definitions may equally apply to the description below.

As shown, the exemplary therapeutic interchange 1100 starts with operation 1102 where a doctor submits a therapeutic interchange. Steps and details relating to operation 1102 may include operations 1104-1110. At operation 1104, preferred medication may be inputted. At operation 1106 a category (or more than one) of the medicine may be selected. At operation 1108, a product(s) (of medicine) may be selected by priority. Further, at operation 1110, special note(s) and/or instruction(s) (from the doctor) may be added. At operation 1110, the doctor may sign and submit the preferred medication. In this manner, doctors may select a preferred medication.

The doctor, per operation 1114, may then create a prescription (RX) for a patient and send it to a fulfillment company (FC). Steps and details relating to operation 1114 may include operations 1116-1118. Per operation 1116, the RX may be submitted via an electronic medical record (EMR) as an electronic prescription (e-RX). Per operation 1118, the FC may capture and/or otherwise receive the RX electronically.

The FC may reach out, per operation 1100, and gather patient info. In this manner, the FC may proactively reach out to the patient to take action. A step and detail relating to operation 1100 may include operation 1102. Per operation 1102, data collection may occur via a phone, a patient app, and/or any other portal (e.g. browser, etc.).

The FC, per operation 1104 may then run the patient's insurance. Steps and details relating to operation 1104 may include operations 1106-1108. Per operation 1106, it is determined if prior authorization is required for coverage. In one embodiment, if prior authorization is required, it is requested. Per operation 1108, it is determined if a coupon is available to lower price. In one embodiment, if a coupon is available, it is applied.

It is then determined, per operation 1132, whether the RX is more than the no more than price of the doctor's therapeutic interchange. In one embodiment, the no more than price may be a pre-determined maximum price indicated by the patient, or a maximum price that the doctor does not want their patient to pay. If the RX is more than the no more price, the interchange continues on to operation 1130 where the FC will then automatically switch to the next preferred product based on the therapeutic interchange.

Once the RX is less than the no more than price, the interchange continues on to operation 1134 where the patient is notified of the product.

In one embodiment, the therapeutic interchange 1100 may follow after a doctor conducts and provides a diagnosis. The doctor may then determine a proper treatment for the diagnosis. As part of the proper treatment, the therapeutic interchange may be used to determine a relevant product which conforms with input(s) by the doctor as well as input(s) by the patient (e.g. including patient attributes, etc.). In this manner, the product selected and presented (per operation 1134) may be dictated by the doctor, but also take into consideration personal attributes and preferences from a patient (e.g. location, price constraint, allergies, lifestyle, etc.).

In other embodiments, the therapeutic interchange 1100 may also allow doctors to rank substitute medications following their preferred medication (per operations 1104-1110). Further, a doctor may authorize the FC to switch the product selected (via operations 1104-1134) based on preferences (of both the doctor and the patient) based on a predetermined ranked order. For example, the doctor may indicate that price is a higher priority over location. The patient may indicate that they have an allergy (e.g. egg, etc.) which may have a higher priority over generic versus brand name. The doctor may receive preferences from the patient and control the overall ranked order of the available preferences.

Additionally, it should be noted that the therapeutic interchange 1100 focuses primarily on the flow from operation 1102 to operation 1114 to operation 1100 to operation 1104 to decision 1132 (which may involve operation 1130) to operation 1134. To that end, the therapeutic interchange 1100 includes an arrow from one operation to the next. In contrast, the sub elements under each operation are intended as details associated with each operation/decision of the flow. Thus, for example, operations 1104-1110 pertain to details of operation 1102, operations 1116-118 pertain to details of operation 1114, etc.

It should be understood that the arrangement of components illustrated in the Figures described are exemplary and that other arrangements are possible. It should also be understood that the various system components (and means) defined by the claims, described below, and illustrated in the various block diagrams represent logical components in some systems configured according to the subject matter disclosed herein.

For example, one or more of these system components (and means) may be realized, in whole or in part, by at least some of the components illustrated in the arrangements illustrated in the described Figures. In addition, while at least one of these components are implemented at least partially as an electronic hardware component, and therefore constitutes a machine, the other components may be implemented in software that when included in an execution environment constitutes a machine, hardware, or a combination of software and hardware.

More particularly, at least one component defined by the claims is implemented at least partially as an electronic hardware component, such as an instruction execution machine (e.g., a processor-based or processor-containing machine) and/or as specialized circuits or circuitry (e.g., discreet logic gates interconnected to perform a specialized function). Other components may be implemented in software, hardware, or a combination of software and hardware. Moreover, some or all of these other components may be combined, some may be omitted altogether, and additional components may be added while still achieving the functionality described herein. Thus, the subject matter described herein may be embodied in many different variations, and all such variations are contemplated to be within the scope of what is claimed.

In the description above, the subject matter is described with reference to acts and symbolic representations of operations that are performed by one or more devices, unless indicated otherwise. As such, it will be understood that such acts and operations, which are at times referred to as being computer-executed, include the manipulation by the processor of data in a structured form. This manipulation transforms the data or maintains it at locations in the memory system of the computer, which reconfigures or otherwise alters the operation of the device in a manner well understood by those skilled in the art. The data is maintained at physical locations of the memory as data structures that have particular properties defined by the format of the data. However, while the subject matter is being described in the foregoing context, it is not meant to be limiting as those of skill in the art will appreciate that various of the acts and operations described hereinafter may also be implemented in hardware.

To facilitate an understanding of the subject matter described herein, many aspects are described in terms of sequences of actions. At least one of these aspects defined by the claims is performed by an electronic hardware component. For example, it will be recognized that the various actions may be performed by specialized circuits or circuitry, by program instructions being executed by one or more processors, or by a combination of both. The description herein of any sequence of actions is not intended to imply that the specific order described for performing that sequence must be followed. All methods described herein may be performed in any suitable order unless otherwise indicated herein or otherwise clearly contradicted by context.

The use of the terms “a” and “an” and “the” and similar referents in the context of describing the subject matter (particularly in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. Furthermore, the foregoing description is for the purpose of illustration only, and not for the purpose of limitation, as the scope of protection sought is defined by the claims as set forth hereinafter together with any equivalents thereof entitled to. The use of any and all examples, or exemplary language (e.g., “such as”) provided herein, is intended merely to better illustrate the subject matter and does not pose a limitation on the scope of the subject matter unless otherwise claimed. The use of the term “based on” and other like phrases indicating a condition for bringing about a result, both in the claims and in the written description, is not intended to foreclose any other conditions that bring about that result. No language in the specification should be construed as indicating any non-claimed element as essential to the practice of the invention as claimed.

The embodiments described herein included the one or more modes known to the inventor for carrying out the claimed subject matter. Of course, variations of those embodiments will become apparent to those of ordinary skill in the art upon reading the foregoing description. The inventor expects skilled artisans to employ such variations as appropriate, and the inventor intends for the claimed subject matter to be practiced otherwise than as specifically described herein. Accordingly, this claimed subject matter includes all modifications and equivalents of the subject matter recited in the claims appended hereto as permitted by applicable law. Moreover, any combination of the above-described elements in all possible variations thereof is encompassed unless otherwise indicated herein or otherwise clearly contradicted by context.

What is claimed is:

1. A non-transitory computer readable storage medium storing one or more programs, the one or more programs comprising instructions which, when executed by an apparatus, cause the apparatus to:
retrieve at least one of historical interaction data of a first patient, or real-time interaction data of the first patient;
based on at least one of the historical interaction data of the first patient or the real-time interaction data of the first patient, present at least one potential diagnosis;
receive a selection of a determined diagnosis from the at least one potential diagnosis by a medical provider;
receive a selection of additional patient attributes by the medical provider; and
present the recommended treatment to the medical provider based, at least in part, on data mining:
at least one of the historical interaction data of the first patient, or the real-time interaction data of the first patient;
the selection of the determined diagnosis; and
the selection of additional patient attributes.

2. The non-transitory computer readable storage medium of claim 1, wherein the historical interaction data of the first patient includes an asynchronous interaction.

3. The non-transitory computer readable storage medium of claim 2, wherein the asynchronous interaction includes at least one of:
patient medical history;
patient and medical provider interaction;
patient medical procedure;
patient medical condition;
patient medical allergy;
family medical procedure;
family medical condition;
family medical allergy;
family health history;
prior medical interaction;
prior medical diagnosis;
prior prescription;
prior illness;
prior surgery;
prior immunization;
results of physical exams;
results of genetic test;
frequency of past screening,
substance abuse;
pregnancy complications;
prior hospitalization;
communication associated with the first patient;
lifestyle habits;
personal data associated with the first patient; or
remote patient monitoring.

4. The non-transitory computer readable storage medium of claim 1, wherein historical interaction data of the first patient includes clinical data and past patient attributes including at least two of:
patient pricing considerations,
patient lifestyle considerations, or
patient availability considerations.

5. The non-transitory computer readable storage medium of claim 1, wherein the real-time interaction data of the first patient includes a synchronous interaction.

6. The non-transitory computer readable storage medium of claim 5, wherein the synchronous interaction includes at least one of:
real-time behavior observations;
real-time communication;
digital communication;
video patient evaluations;
health triage;
in-person visit;
live examination;
telehealth communication;
in-house provider visit;
in-office provider visit; or
current vitals of the first patient.

7. The non-transitory computer readable storage medium of claim 1, wherein the historical interaction data further includes at least one of:
medical data associated with a clinical study,
real-world medically relevant evidence including at least one of population data, specific region data, or specific clinic data, or
availability of a prescription based on a location of pharmacy, pharmacy charges, manufacturer's rebate, or insurance payment.

8. The non-transitory computer readable storage medium of claim 1, wherein the one or more programs comprises instructions which, when executed by the apparatus, cause the apparatus to anonymize the historical interaction of the first patient, the real-time interaction of the first patient, and the determined diagnosis.

9. The non-transitory computer readable storage medium of claim 8, wherein the one or more programs comprises instructions which, when executed by the apparatus, cause the apparatus to, based on at least one of the anonymized historical interaction of the first patient, the anonymized real-time interaction of the first patient, or the anonymized determined diagnosis, extract relevant medical data from one or more clinical databases.

10. The non-transitory computer readable storage medium of claim 9, wherein the relevant medical data is used in presenting the at least one potential diagnosis.

11. The non-transitory computer readable storage medium of claim 1, wherein the real-time interaction data of the first patient includes an update to at least aspect of the historical interaction data of the first patient.

12. The non-transitory computer readable storage medium of claim 1, wherein the additional patient attributes include one or more attributes associated with the first patient and the determined diagnosis.

13. The non-transitory computer readable storage medium of claim 12, wherein the one or more attributes include at least one of a maximum time to receive a prescription, a location to receive the prescription, or insurance constraints.

14. The non-transitory computer readable storage medium of claim 1, wherein the recommended treatment is updated based on a change to at least one of the historical interaction data of the first patient, the real-time interaction data of the first patient, or the additional patient attributes.

15. The non-transitory computer readable storage medium of claim 1, wherein automatically data mining includes assigning a ranking associated with each of the historical interaction data of the first patient, the real-time interaction data of the first patient, and the additional patient attributes.

16. The non-transitory computer readable storage medium of claim 1, wherein the one or more programs comprises instructions which, when executed by the apparatus, cause the apparatus to automatically prepare an order based on the recommended treatment.

17. The non-transitory computer readable storage medium of claim 16, wherein the one or more programs comprises instructions which, when executed by the apparatus, cause the apparatus to ship the order to the first patient and track the shipping of the order to the first patient.

18. The non-transitory computer readable storage medium of claim 1, wherein the recommended treatment to the medical provider is further presented based on input from an artificial intelligence system that analyzes at least two of:
the historical interaction data of the first patient,
the real-time interaction data of the first patient;
the selection of the determined diagnosis; or
the selection of additional patient attributes.

19. A computer-implemented method, comprising:
retrieving, using a processor, at least one of historical interaction data of a first patient, or real-time interaction data of the first patient;
based on at least one of the historical interaction data of the first patient or the real-time interaction data of the first patient, presenting, using the processor, at least one potential diagnosis;
receiving, using the processor, a selection of a determined diagnosis from the at least one potential diagnosis by a medical provider;
receiving, using the processor, a selection of additional patient attributes by the medical provider; and
presenting, using the processor, a recommended treatment to the medical provider based, at least in part, on data mining:
at least one of the historical interaction data of the first patient, or the real-time interaction data of the first patient;
the selection of the determined diagnosis; and
the selection of additional patient attributes.

20. A device, comprising:
a non-transitory memory storing instructions; and
one or more processors in communication with the non-transitory memory, wherein the one or more processors execute the instructions to:
retrieve at least one of historical interaction data of a first patient, or real-time interaction data of the first patient;
based on at least one of the historical interaction data of the first patient or the real-time interaction data of the first patient, present at least one potential diagnosis;
receive a selection of a determined diagnosis from the at least one potential diagnosis by a medical provider;
receive a selection of additional patient attributes by the medical provider;
present a recommended treatment to the medical provider based, at least in part, on data mining:
at least one of the historical interaction data of the first patient, or the real-time interaction data of the first patient;
the selection of the determined diagnosis; and
the selection of additional patient attributes.
